(19) United States
(12) Patent Application Publication
(10) Pub. No.: US 2026/0029406 A1
(43) Pub. Date: Jan. 29, 2026

(54) PROTEIN IDENTIFICATION VIA PEPTIDE BARCODES

(71) Applicant: Quantum-Si Incorporated, Branford, CT (US)

(72) Inventor: Brian Reed, Madison, CT (US)

(73) Assignee: Quantum-Si Incorporated, Branford, CT (US)

(21) Appl. No.: 19/284,284

(22) Filed: Jul. 29, 2025

Related U.S. Application Data
(60) Provisional application No. 63/676,633, filed on Jul. 29, 2024.

(51) Int. Cl. G01N 33/68; C12N 15/10; C40B 20/04

(52) U.S. Cl. CPC G01N 33/6845; C12N 15/1065; C40B 20/04

(57) ABSTRACT

The present disclosure relates to methods and compositions for protein identification via peptide barcodes.

### CROSS REFERENCE TO RELATED APPLICATIONS

This application claims priority under 35 U.S.C. § 119(e) to U.S. Provisional Application No. 63/676,633, filed Jul. 29, 2024, which is hereby incorporated by reference in its entirety.

### REFERENCE TO AN ELECTRONIC SEQUENCE LISTING

The contents of the electronic sequence listing (R070870177US01-SEQ-KVC.xml; Size: 4,857 bytes; and Date of Creation: Jul. 28, 2025) are herein incorporated by reference in its entirety.

### BACKGROUND

Proteins are the main structural and functional components of cells, driving key biological and cellular processes. Next-generation DNA sequencing technologies have revolutionized our understanding of heredity and gene regulation, but the complex and dynamic states of cells are not fully captured by the genome and transcriptome. Applying similar approaches to proteomics has been difficult because of the scale, dynamic range, and inability to amplify the source.

### SUMMARY

Aspects of the present disclosure related to methods and compositions for protein identification via peptide barcodes.

Aspects of the present disclosure relate to a composition comprising: a first affinity reagent attached to a first peptide tag; and a second affinity reagent attached to a second peptide tag; wherein the first affinity reagent and the second affinity reagent are each configured to bind a target analyte to form a complex comprising the first affinity reagent, the second affinity reagent, and the target analyte.

In some embodiments, the first affinity reagent is directly attached to the first peptide tag. In some embodiments, the second affinity reagent is directly attached to the second peptide tag. In some embodiments, the first affinity reagent is attached to the first peptide tag via a linker. In some embodiments, the second affinity reagent is attached to the second peptide tag via a linker.

In some embodiments, the linker is a peptide linker or an oligonucleotide linker.

In some embodiments, the target analyte is a target protein. In some embodiments, the target protein is a monomeric or multimeric protein.

In some embodiments, the first affinity reagent and the second affinity reagent are each configured to bind different sites on the target analyte. In some embodiments, the first affinity reagent and the second affinity reagent are each configured to bind different epitopes on the target protein.

In some embodiments, the first affinity reagent is an antibody, a nanobody, an aptamer, or an adaptor protein. In some embodiments, the second affinity reagent is an antibody, a nanobody, an aptamer, or an adaptor protein.

In some embodiments, the first peptide tag comprises a first cleavage site, one or more first peptide barcodes, and/or a first functional moiety. In some embodiments, the second peptide tag comprises a second cleavage site, one or more second peptide barcodes, and/or a second functional moiety.

In some embodiments, the one or more first peptide barcodes comprise a reagent barcode, a unique molecular identifier, a sample barcode, or any combination thereof. In some embodiments, the one or more second peptide barcodes comprise a reagent barcode, a unique molecular identifier, a sample barcode, or any combination thereof.

In some embodiments, the first functional moiety is a click chemistry handle, an enzyme, or an enzyme substrate. In some embodiments, the second functional moiety is a click chemistry handle, an enzyme, or an enzyme substrate.

In some embodiments, modification of the first peptide tag and/or the second peptide tag is indicative of formation of the complex.

In some embodiments, the first peptide tag comprises a first functional moiety. In some embodiments, the first functional moiety chemically modifies the second peptide tag. In some embodiments, the first functional moiety chemically modifies the second peptide tag in the presence of the target analyte. In some embodiments, the first functional moiety chemically modifies the second peptide tag when the first affinity reagent and the second affinity reagent are each bound to the target analyte. In some embodiments, the first functional moiety chemically modifies an amino acid residue on the second peptide tag. In some embodiments, the first functional moiety cleaves one or more amino acid residues from the second peptide tag. In some embodiments, the first functional moiety comprises an enzyme, and the second peptide tag comprises a substrate of the enzyme. In some embodiments, the second peptide tag comprises a second functional moiety. In some embodiments, the first functional moiety comprises an enzyme, and the second functional moiety comprises a substrate of the enzyme. In some embodiments, the first functional moiety and the second functional moiety comprise complementary click chemistry handles.

Aspects of the present disclosure relate to a composition, comprising an affinity reagent attached to a peptide tag, wherein the affinity reagent is configured to bind a target analyte.

In some embodiments, the target analyte is a target protein. In some embodiments, the target protein is a monomeric or a multimeric protein.

In some embodiments, the affinity reagent is an antibody, a nanobody, an aptamer, or an adaptor protein. In some embodiments, the peptide tag comprises a cleavage site and one or more peptide barcodes.

In some embodiments, the one or more peptide barcodes comprise a reagent barcode, a unique molecular identifier, a sample barcode, or any combination thereof.

Aspects of the present disclosure relate to a complex, comprising: a first affinity reagent attached to a first peptide tag; and a second affinity reagent attached to a second peptide tag; wherein the first affinity reagent and the second affinity reagent are each bound to a target analyte.

In some embodiments, the first affinity reagent is directly attached to the first peptide tag. In some embodiments, the second affinity reagent is directly attached to the second peptide tag. In some embodiments, the first affinity reagent is attached to the first peptide tag via a linker. In some embodiments, the second affinity reagent is attached to the second peptide tag via a linker.

In some embodiments, the linker is a peptide linker or an oligonucleotide linker.

In some embodiments, the target analyte is a target protein. In some embodiments, the target protein is a monomeric or a multimeric protein.

In some embodiments, the first affinity reagent and the second affinity reagent are each configured to bind different sites on the target analyte. In some embodiments, the first affinity reagent and the second affinity reagent are each bound to different epitopes on the target protein. In some embodiments, the first affinity reagent is an antibody, a nanobody, an aptamer, or an adaptor protein. In some embodiments, the second affinity reagent is an antibody, a nanobody, an aptamer, or an adaptor protein.

In some embodiments, the first peptide tag comprises a first cleavage site, one or more first peptide barcodes, and/or a first functional moiety. In some embodiments, the second peptide tag comprises a second cleavage site, one or more second peptide barcodes, and/or a second functional moiety.

In some embodiments, the one or more first peptide barcodes comprise a reagent barcode, a unique molecular identifier, a sample barcode, or any combination thereof. In some embodiments, the one or more second peptide barcodes comprise a reagent barcode, a unique molecular identifier, a sample barcode, or any combination thereof.

In some embodiments, the first functional moiety is a click chemistry handle, an enzyme, or an enzyme substrate. In some embodiments, the second functional moiety is a click chemistry handle, an enzyme, or an enzyme substrate.

In some embodiments, the first peptide tag comprises a first functional moiety.

In some embodiments, the first functional moiety chemically modifies the second peptide tag. In some embodiments, the first functional moiety chemically modifies the second peptide tag in the presence of the target analyte. In some embodiments, the first functional moiety chemically modifies the second peptide tag when the first affinity reagent and the second affinity reagent are each bound to the target analyte. In some embodiments, the first functional moiety chemically modifies an amino acid residue on the second peptide tag. In some embodiments, the first functional moiety cleaves one or more amino acid residues from the second peptide tag. In some embodiments, the first functional moiety comprises an enzyme, and the second peptide tag comprises a substrate of the enzyme.

In some embodiments, the second peptide tag comprises a second functional moiety.

In some embodiments, the first functional moiety comprises an enzyme, and the second functional moiety comprises a substrate of the enzyme. In some embodiments, the first functional moiety and the second functional moiety comprise complementary click chemistry handles.

In some embodiments, the first peptide tag is attached to the second peptide tag. In some embodiments, the first peptide tag is covalently attached to the second peptide tag. In some embodiments, the first peptide tag is non-covalently attached to the second peptide tag.

Aspects of the present disclosure relate to a method of identifying a target analyte, comprising: (i) contacting a sample with one or more analyte-specific reagent sets, each analyte-specific reagent set comprising a first affinity reagent attached to a first peptide tag and a second affinity reagent attached to a second peptide tag, wherein the first affinity reagent and the second affinity reagent are each configured to bind a target analyte; (ii) contacting the sample with a cleaving agent, wherein the cleaving agent removes the first peptide tag from the first affinity reagent and/or the second peptide tag from the second affinity reagent; and (iii) sequencing at least a portion of the first peptide tag and/or at least a portion of the second peptide tag.

In some embodiments, the first affinity reagent is directly attached to the first peptide tag. In some embodiments, the second affinity reagent is directly attached to the second peptide tag. In some embodiments, the first affinity reagent is attached to the first peptide tag via a linker. In some embodiments, the second affinity reagent is attached to the second peptide tag via a linker.

In some embodiments, the linker is a peptide linker or an oligonucleotide linker.

In some embodiments, the method further comprises isolating the first peptide tag and/or the second peptide tag from the sample prior to sequencing.

In some embodiments, the first affinity reagent and the second affinity reagent each bind to different sites on the target analyte to form a complex comprising the first affinity reagent, the second affinity reagent, and the target analyte.

In some embodiments, the target analyte is a target protein. In some embodiments, the target protein is a monomeric or multimeric protein.

In some embodiments, the first affinity reagent is an antibody, nanobody, aptamer, or adaptor protein. In some embodiments, the second affinity reagent is an antibody, nanobody, aptamer, or adaptor protein.

In some embodiments, the first peptide tag comprises a first cleavage site, one or more first peptide barcodes, and/or a first functional moiety.

In some embodiments, sequencing comprises determining one or more chemical characteristics of the one or more first peptide barcodes.

In some embodiments, the second peptide tag comprises a second cleavage site, one or more second peptide barcodes, and/or a second functional moiety.

In some embodiments, sequencing comprises determining one or more chemical characteristics of the one or more second peptide barcodes.

In some embodiments, the method further comprises contacting the sample with a peptide ligase.

In some embodiments, the peptide ligase ligates the first peptide tag to the second peptide tag. In some embodiments, the peptide ligase ligates the first peptide tag to the second peptide tag in the presence of the target analyte. In some embodiments, the peptide ligase ligates the first peptide tag to the second peptide tag when the first affinity reagent and the second affinity reagent are each bound to the target analyte.

In some embodiments, sequencing comprises determining one or more chemical characteristics of a ligation product comprising at least a portion of the first peptide tag and at least a portion of the second peptide tag.

In some embodiments, the one or more first peptide barcodes comprise an affinity reagent barcode, a unique molecular identifier, a sample barcode, or any combination thereof. In some embodiments, the one or more second peptide barcodes comprise an affinity reagent barcode, a unique molecular identifier, a sample barcode, or any combination thereof.

In some embodiments, the first functional moiety is a click chemistry handle, an enzyme, or an enzyme substrate. In some embodiments, the second functional moiety is a click chemistry handle, an enzyme, or an enzyme substrate.

In some embodiments, the first affinity reagent binds to a first binding site on the target analyte in the sample and the second affinity reagent binds to a second binding site on the target analyte in the sample.

In some embodiments, the first functional moiety binds to the second functional moiety when the first affinity reagent is bound to the first binding site on the target analyte and the second affinity reagent is bound to the second binding site on the target analyte. In some embodiments, the first functional moiety chemically modifies the second peptide tag. In some embodiments, the first functional moiety chemically modifies the second peptide tag in the presence of the target analyte. In some embodiments, the first functional moiety chemically modifies the second peptide tag when the first affinity reagent and the second affinity reagent are each bound to the target analyte. In some embodiments, the first functional moiety chemically modifies an amino acid residue on the second peptide tag. In some embodiments, first functional moiety cleaves one or more amino acid residues from the second peptide tag. In some embodiments, the first functional moiety comprises an enzyme, and the second peptide tag comprises a substrate of the enzyme.

In some embodiments, the second peptide tag comprises a second functional moiety.

In some embodiments, the first functional moiety comprises an enzyme, and the second functional moiety comprises a substrate of the enzyme. In some embodiments, the first functional moiety and the second functional moiety comprise complementary click chemistry handles.

In some embodiments, sequencing comprises: monitoring a signal for signal pulses corresponding to interactions between one or more amino acid recognizers and a peptide; and determining at least one chemical characteristic of the peptide based on a characteristic pattern in the signal.

In some embodiments, (i) comprises: contacting the sample with a plurality of analyte-specific reagent sets. In some embodiments, the plurality of analyte-specific reagent sets comprises at least 1,000, at least 2,000, at least 3,000, at least 4,000, or at least 5,000 analyte-specific reagent sets.

In some embodiments, the sample comprises a panel of target analytes. In some embodiments, the panel of target analytes comprises at least 1,000, at least 2,000, at least 3,000, at least 4,000, or at least 5,000 target analytes. In some embodiments, the panel of target analytes comprises about 5,000 target analytes.

Aspects of the present disclosure relate to a method of identifying a target analyte in a sample, comprising contacting a sample with any composition described herein.

In some embodiments, the method further comprises isolating the first peptide tag and/or the second peptide tag from the sample. In some embodiments, the method further comprises separating the one or more first peptide barcodes from the first peptide tag and the one or more second peptide barcodes from the second peptide tag. In some embodiments, the method further comprises sequencing the one or more first peptide barcodes and/or the one or more second peptide barcodes to identify a target analyte in the sample.

Aspects of the present disclosure relate to a system, comprising: at least one hardware processor; and at least one non-transitory computer-readable storage medium storing processor-executable instructions that, when executed by the at least one hardware processor, cause the at least one hardware processor to perform any method described herein.

Aspects of the present disclosure relate to at least one non-transitory computer-readable storage medium storing processor-executable instructions that, when executed by at least one hardware processor, cause the at least one hardware processor to perform any method described herein.

Aspects of the present disclosure relate to a kit, comprising materials and reagents for carrying out any method described herein.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute part of this specification, illustrate several embodiments of the disclosure and together with the accompanying description, serve to explain the principles of the disclosure.

FIG. 1A shows an example configuration of an affinity reagent attached to a peptide tag in accordance with certain aspects of the disclosure.

FIG. 1B schematically illustrates example processes of peptide-based proximity methods in accordance with certain aspects of the disclosure.

FIG. 2 shows an example overview of real-time dynamic protein sequencing. Protein samples are digested into peptide fragments, immobilized in nanoscale reaction chambers, and incubated with a mixture of freely diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs).

### DETAILED DESCRIPTION

Proximity ligation is a method of protein identification in which two antibodies, each attached to a different oligonucleotide, bind to a target protein, if present, at which point the two different oligonucleotides interact to form a complex. A fluorophore is often used to bind to the oligonucleotides of the complex. If the target protein is present, the fluorophore binds to the complex and fluoresces, thus indicating the presence of the target protein.

Traditional proximity ligation is limited in use due to difficulties associated with attaching oligonucleotides to antibodies and because the readout is restricted to the number of fluorophores that can be used in a single sample (i.e., only a small number of target proteins can be detected within a sample). In addition, because DNA is limited to four nucleobases, DNA tags must be lengthy to encode sufficient information to distinguish large panels of antibodies. Methods of DNA sequencing have been used in the context of proximity ligation, but DNA sequencing itself is limited due to oligonucleotide amplification errors and challenges associated with in situ DNA sequencing.

Accordingly, the present disclosure relates to a method of proximity ligation in which peptides, and not oligonucleotides, are used to indicate whether a target protein is present in a sample. Peptide-based proximity ligation is an improvement over current methods because peptides provide for a broader range of modifications for downstream analysis and peptides are amenable to in situ sequencing. Unlike traditional proximity ligation, which requires isolation of an antibody and subsequent attachment of an oligonucleotide to the antibody, peptide-based proximity ligation does not require use of an oligonucleotide, so an affinity reagent (e.g., an antibody) can be expressed with an attached peptide tag without additional chemical steps or the introduction of impurities. In addition, a short peptide tag can encode sufficient information for use in large panels of antibodies due to the availability of twenty amino acids, as opposed to four nucleobases.

Aspects of the present disclosure relate, at least in part, to methods and compositions for protein identification via peptide barcodes. Protein identification is the process by which the presence or absence of a protein within a sample (e.g., a patient sample, a cell culture, a tissue section, a panel) is determined. In some embodiments, protein identification is useful for determining the presence or absence of a protein within a sample. In some embodiments, a sample is a patient sample. In some embodiments, a sample is a cell culture. In some embodiments, a sample is a tissue section. In some embodiments, a sample is a panel. In some embodiments, a panel comprises at least 100, at least 200, at least 300, at least 400, at least 500, at least 600, at least 700, at least 800, at least 900, at least 1,000, at least 2,000, at least 3,000, at least 4,000, or at least 5,000 proteins. In some embodiments, a panel comprises many thousands of proteins. In the context of a patient sample, the presence or absence of a protein can be indicative of a disease state. Improved methods of protein identification can lead to improved diagnosis of disease or an acceleration in biotechnology research.

Aspects of the present disclosure relate to the use of peptide-based proximity ligation to identify one or more proteins in a sample. Peptide-based proximity ligation is a method in which one or more affinity reagents (e.g., a molecule that binds to a target analyte), attached to a peptide tag, binds to a target analyte. In some embodiments, one affinity reagent binds to a target analyte within a sample and the presence of the target analyte is determined by sequencing a peptide tag attached to the affinity reagent. In some embodiments, two affinity reagents, each attached to different peptide tags, bind to different sites on a target analyte, at which point the two affinity reagents interact with each other and the presence of the target analyte is determined by sequencing one or both of the peptide tags. In some embodiments, multiple sets of two affinity reagents are added to a sample to detect the presence or absence of multiple different target analytes. In general, peptide-based proximity ligation is a method of determining the presence of a target analyte within a sample by contact the sample with one or more affinity reagents attached to one or more peptide tags, allowing the one or more affinity reagents to bind a target analyte within a sample, then sequencing the one or more peptide tags, or a portion thereof, to determine whether the target analyte is present within the sample.

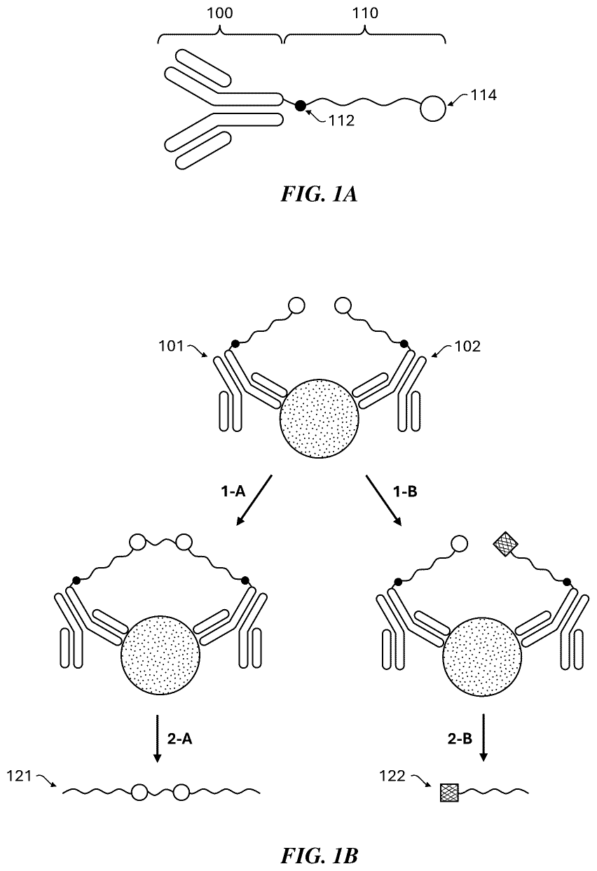

FIG. 1A depicts an example configuration of an analyte-specific reagent comprising an affinity reagent 100 attached to a peptide tag 110. In some embodiments, affinity reagent 100 is a molecule (e.g., a biomolecule) configured to bind a target analyte. In some embodiments, affinity reagent 100 is an antibody, an antigen-binding portion of an antibody (e.g., a single-chain antibody variable fragment (scFv) or V_{H}H fragment), or an aptamer. In some embodiments, affinity reagent 100 is a protein, such as an antibody. In some such embodiments, affinity reagent 100 and peptide tag 110 can be expressed as a single polypeptide fusion molecule from an expression construct encoding both components. In some embodiments, affinity reagent 100 and peptide tag 110 can be produced separately (e.g., by expression, synthesis, or other methodologies known in the art) and conjugated by covalent or non-covalent attachment means described herein or known in the art.

In some embodiments, peptide tag 110 comprises a peptide barcode, which can include an amino acid sequence indicative of the affinity reagent to which the peptide tag is attached. In some embodiments, peptide tag 110 comprises a cleavage site 112 and a functional moiety 114. In some embodiments, cleavage site 112 is located between a peptide barcode of the peptide tag and the site of attachment between the peptide tag and the affinity reagent. In this way, cleavage at the cleavage site separates the peptide tag from the affinity reagent while retaining the barcode information in the peptide tag (e.g., for analysis by sequencing). Accordingly, in some embodiments, the peptide barcode of a peptide tag is located between cleavage site 112 and functional moiety 114. In some embodiments, functional moiety 114 can form part of the peptide barcode of a peptide tag. As described herein, in some embodiments, functional moiety 114 is configured to interact with a functional moiety of another analyte-specific reagent of the same analyte-specific reagent set.

FIG. 1B schematically illustrates example processes involving the use of analyte-specific reagents in accordance with certain aspects of the disclosure. In some embodiments, methods of the disclosure comprise contacting a sample with one or more analyte-specific reagent sets, where each analyte-specific reagent set is configured to bind to a target analyte. As shown, in some embodiments, the sample is contacted with an analyte-specific reagent set that comprises a first analyte-specific reagent 101 and a second analyte-specific reagent 102. In some embodiments, the first and second analyte-specific reagents bind to different sites on a target analyte (shown as stippled shape). In some embodiments, the target analyte is a target protein, and the first and second analyte-specific reagents bind to different epitopes on the target protein.

In some embodiments, first analyte-specific reagent 101 comprises a first peptide tag comprising a first functional moiety, and second analyte-specific reagent 102 comprises a second peptide tag comprising a second functional moiety. The peptide-based proximity methods of the disclosure include different implementations in which the proximity of analyte-bound reagents of a set permits interaction between functional moieties of the respective peptide tags. This interaction can result in a chemical change to one or both peptide tags (e.g., one or both functional moieties of the peptide tags), and sequencing of the peptide tag(s) permits the identification of proximity binding and thus the identification of target analyte in the sample.

As shown by process 1-A, in some embodiments, the first and second functional moieties are configured for ligation by chemical or enzymatic means described herein. For example, in some embodiments, the first and second functional moieties comprise complementary click chemistry handles. In some such embodiments, process 1-A comprises subjecting the sample to reaction conditions (e.g., change in temperature, addition of catalyst, etc.) suitable for the complementary click chemistry handles to react and form a covalent attachment between peptide tags when the first and second functional moieties are in proximity.

In some embodiments, the first and second functional moieties comprise enzymatically ligatable amino acids, and process 1-A comprises contacting the sample with a peptide ligase that forms a covalent attachment between the ligatable amino acids when the first and second functional moieties are in proximity. By way of example and not limitation, in some embodiments, the first functional moiety comprises a sortase recognition sequence, and the second functional moiety comprises a polyglycine sequence. In the presence of a sortase enzyme (e.g., sortase A), the enzyme mediates a ligation reaction between the sortase recognition sequence and the polyglycine sequence, thereby forming a covalent attachment between peptide tags when the first and second functional moieties are in proximity. Further examples of ligases and ligatable amino acids are described herein and known in the art.

In some embodiments, following the chemical or enzymatic ligation of process 1-A, the sample is contacted with one or more cleaving agents (e.g., one or more endopeptidases) in process 2-A. The one or more cleaving agents are configured to bind and cleave at the cleavage sites of the first and second peptide tags. In some embodiments, the cleavage sites of the first and second peptide tags comprise the same amino acid sequence and are capable of being cleaved by the same type of cleaving agent. In some embodiments, the cleavage sites of the first and second peptide tags comprise different amino acid sequences that are cleaved by different cleaving agents. As shown, where the proximity of analyte-bound reagents results in ligation of the first and second peptide tags, the cleavage of process 2-A produces a ligated polypeptide 121 comprising amino acid sequences (e.g., peptide barcodes) of both peptide tags. In some embodiments, ligated polypeptide 121 is analyzed (e.g., by peptide sequencing), and the identification of amino acid sequences of both peptide tags is indicative of proximity binding and thus the presence of the target analyte in the sample.

As shown by process 1-B, in some embodiments, the first and second functional moieties are configured for chemical modification that does not involve peptide tag ligation. For example, in some embodiments, the first functional moiety of the first peptide tag comprises an enzyme, and the second functional moiety of the second peptide tag comprises a substrate of the enzyme. In some embodiments, the enzyme comprises a peptidase, and the substrate comprises one or more amino acids capable of being cleaved by the peptidase. In some embodiments, the enzyme comprises a transferase (e.g., a kinase, methyltransferase, or other such enzymes described herein and known in the art), and the substrate comprises a site capable of modification by the transferase. In some embodiments, process 1-B comprises subjecting the sample to reaction conditions (e.g., change in temperature, addition of cofactor, etc.) suitable for the enzyme to react with the substrate to chemically modify the second peptide tag when the first and second functional moieties are in proximity.

In some embodiments, following the chemical modification of process 1-B, the sample is contacted with one or more cleaving agents (e.g., one or more endopeptidases) in process 2-B. In some embodiments, the one or more cleaving agents include at least one cleaving agent configured to bind and cleave at the cleavage site of the second peptide tag. The implementation illustrated by processes 2-A and 2-B may not require cleavage of the first peptide tag and thus, in some embodiments, the presence of a cleavage site in the first peptide tag is optional. As shown, where the proximity of analyte-bound reagents results in chemical modification of the second peptide tag, the cleavage of process 2-B produces a modified polypeptide 122 comprising one or more chemical changes relative to the second peptide tag prior to the chemical modification of process 1-B. In some embodiments, modified polypeptide 122 is analyzed (e.g., by peptide sequencing), and the identification of the one or more chemical changes in the second peptide tag is indicative of proximity binding and thus the presence of the target analyte in the sample.

In some embodiments, the term proximity refers to the spatial relationship between two elements, such as the distance between two binding sites on a target analyte or the distance between two affinity reagents bound to a target analyte. In some embodiments, as described elsewhere herein, two affinity reagents (affinity reagent pair) comprising peptide tags form a complex with a target analyte, and the peptide tags remain in sufficient proximity to interact with each other when the affinity reagents are bound to the target analyte.

### Compositions and Complexes

Aspects of the present disclosure relate to a composition for use in peptide-based proximity ligation. As described herein, peptide-based proximity ligation is a process in which one or more affinity reagents, each attached to a peptide tag, bind to a target analyte in a sample. If the one or more affinity reagents bind to a target analyte within a sample, the peptide tags attached to the one or more affinity reagents can be analyzed to determine the identity of the target analyte.

### Affinity Reagents

Accordingly, aspects of the present disclosure relate, at least in part, to a composition comprising a first affinity reagent attached to a first peptide tag and a second affinity reagent attached to a second peptide tag. In some embodiments, the term “affinity reagent” refers to a molecule that is configured to bind to a target analyte. In some embodiments, an affinity reagent is an antibody. In some embodiments, an affinity reagent is a nanobody. In some embodiments, an affinity reagent is a monoclonal antibody. In some embodiments, an affinity reagent is a recombinant antibody. In some embodiments, an affinity reagent is a synthetic antibody. In some embodiments, an affinity reagent is a single domain antibody. In some embodiments, an affinity reagent is a fragment of an antibody. In some embodiments, an affinity reagent is a single-chain variable fragment (scFv). In some embodiments, an affinity reagent is an antigen-binding fragment (Fab). In some embodiments, an affinity reagent is an aptamer. In some embodiments, an affinity reagent is an adaptor protein. In some embodiments, a first affinity reagent is an antibody and a second affinity reagent is an antibody. In some embodiments, a first affinity reagent is a nanobody and a second affinity reagent is an antibody. In some embodiments, a first affinity reagent is an aptamer and a second affinity reagent is an antibody. In some embodiments, a first affinity reagent is an adaptor protein and a second affinity reagent is an antibody.

The affinity reagents of the present disclosure are configured to bind to a site on a target analyte. In some embodiments, a first affinity reagent is an antibody and a second affinity reagent is an antibody. In some embodiments, upon binding to a target analyte, the affinity reagents form a complex comprising a first affinity reagent, a second affinity reagent, and the target analyte.

Aspects of the present disclosure relate to a first affinity reagent that binds to a first binding site on a target analyte and a second affinity reagent that binds to a second binding site on the target analyte. In some embodiments, the binding of a first affinity reagent to a first binding site on a target analyte and a second affinity reagent to a second binding site on the target analyte results in the formation of a complex comprising the first affinity reagent, the second affinity reagent, and the target analyte. The formation of a complex is, in some embodiments, indicative of the presence of the target analyte in a sample. In some embodiments, a binding site is an epitope. The term “epitope,” as used herein, includes the specific part of an antigen that is recognized by molecules of the immune system (e.g., antibodies). In some embodiments, an epitope is linear. In some embodiments, an epitope is conformational. As will be appreciated by the skilled artisan, a target analyte that is proteinaceous often has a plurality of antigens which often have a plurality of epitopes. Accordingly, aspects of the present disclosure relate to the identification of affinity reagents that bind to specific epitopes on specific target analytes. Aspects of the present disclosure also relate to the identification of affinity reagent pairs. The term “affinity reagent pairs” includes two separate affinity reagents that bind to different epitopes on the same target analyte. In some embodiments, the binding of two different affinity reagents to different epitopes of the same target analyte and the subsequent formation of a complex including both affinity reagents and the target analyte is indicative of the presence of the target analyte in a sample.

Aspects of the present disclosure relate to the identification of affinity reagents that bind to specific target analytes. In some embodiments, an affinity reagent is a binding partner for a target analyte. In some embodiments, an affinity reagent is specific to a target analyte (i.e., has a high binding affinity for a target analyte). In some embodiments, an affinity reagent is a binding partner for a target analyte and binds to the target analyte with greater affinity and/or specificity than to other components in a sample. Accordingly, an affinity reagent for use in a method of the present disclosure for binding to a target analyte may be selected to have a high binding affinity for the target analyte. In some embodiments, a high binding affinity refers to a binding affinity characterized by a dissociation rate of binding (K_{D}) of less than about 10⁻⁴ M, less than about 10⁻⁵ M, less than about 10⁻⁶ M, less than about 10⁻⁷ M, less than about 10⁻⁸ M, or less than about 10⁻⁹ M. In some embodiments, a high binding affinity refers to a binding affinity characterized by a dissociation rate of binding (K_{D}) of between about 10⁻⁴ M and about 10⁻⁹ M, between about 10⁻⁶ M and about 10⁻¹² M, or between about 10⁻⁶ M and about 10⁻⁹ M.

Methods of screening affinity reagents to identify affinity reagents with high binding affinities to a target analyte are well-understood in the art. In some embodiments, affinity reagents that bind to a target analyte are identified via affinity-based screening. The term “affinity-based screening” includes a method that uses protein display techniques to select antibodies that bind to specific antigens. In some embodiments, affinity reagents that bind to a target analyte are identified via high-throughput antibody screening. The term “high-throughput antibody screening” includes the use of advanced laboratory techniques, automation, and stringent validation processes to rapidly assess large libraries of candidates. High-throughput antibody screening often employs assays such as flow cytometry, microarray analysis, and cell-based assays. Any method known in the art useful for the identification of affinity reagents that bind to target analytes may be used herein. Similarly, methods described herein useful for the identification of affinity reagents that bind to target analytes can be used to identify affinity reagent pairs (i.e., two separate affinity reagents that bind to different epitopes on the same target analyte). In some embodiments, a technique that includes binding a first affinity reagent to a target analyte followed by binding a second affinity reagent to the target analyte is used to identify affinity reagent pairs. Techniques such as the Sandwich ELISA (sELISA), for example, may be used to identify affinity reagent pairs. In some embodiments, affinity reagents pairs are modeled computationally to identify antigen binding sites that are predicted to bind with high affinity to a target analyte. In some embodiments, computational models of target analyte binding by an affinity reagent or affinity reagent pair are validated experimentally using any technique known in the art.

Aspects of the present disclosure relate, at least in part, to a composition comprising an affinity reagent attached to a peptide tag. In some embodiments, only one affinity reagent is required to identify a target analyte within a sample. If a target analyte is present within a sample, a single affinity reagent can bind to the target analyte, at which point the peptide tag attached to the affinity reagent can be analyzed to determine the identity of the target analyte.

Aspects of the present disclosure relate, at least in part, to a complex comprising a first affinity reagent attached to a first peptide tag, a second affinity reagent attached to a second peptide tag, and a target analyte. If a target analyte is present within a sample, then a first affinity reagent and a second affinity reagent can form a complex in which both affinity reagents are bound to the target analyte. A complex only forms if the target analyte is present within a sample and both affinity reagents bind to the target analyte. If a complex forms, then one or both peptide tags attached to the affinity reagents can be analyzed to determine the identity of the target analyte.

### Target Analytes

In some embodiments, the term “target analyte” as used herein, refers to a molecule within a sample. The present disclosure relates to the use of peptide-based proximity ligation to determine the presence or absence of a target analyte within a sample. In some embodiments, the present disclosure relates to the identification of a target analyte within a sample. Determining the presence or absence of a target analyte within a sample can be useful for a variety of purposes, including, inter alia, medical diagnosis, biotechnology research, and tracking therapeutic efficacy. In some embodiments, a target analyte is a target protein. In some embodiments, a target protein is a monomeric protein. In some embodiments, a target protein is a multimeric protein. In some embodiments, a target analyte is a peptide. In some embodiments, a target analyte comprises a plurality of epitopes. A target protein within a sample can be a receptor, a structural protein, a signaling protein, or any other protein found within a biological system. In the event that a target analyte is a target protein and the affinity reagents are antibodies, the affinity reagents bind to different epitopes on the target protein. In some embodiments, a first affinity reagent and a second affinity reagent are each configured to bind different epitopes on a target protein. In some embodiments, a first affinity reagent and a second affinity reagent are bound to different epitopes on a target protein, thus forming a complex with the target protein. In some embodiments, a target analyte is in a panel of target analytes. In some embodiments, a panel of target analytes comprises at least 100, at least 200, at least 300, at least 400, at least 500, at least 600, at least 700, at least 800, at least 900, at least 1,000, at least 2,000, at least 3,000, at least 4,000, or at least 5,000 target analytes. In some embodiments, a panel comprises several thousand target analytes.

### Samples

Aspects of the present disclosure relate, at least in part, to determining the presence or absence of a target analyte within a sample. The present disclosure provides compositions and methods for peptide-based proximity ligation, which can be useful for, inter alia, medical diagnosis, biotechnology research, and tracking therapeutic efficacy. Accordingly, a sample can be any biological sample that contains proteins or peptides. In some embodiments, a sample is a human sample. In some embodiments, a sample is a tissue section. In some embodiments, a sample is blood, blood fraction, blood plasma, blood serum, mucus, cerebrospinal fluid, tears, or any other human sample. In some embodiments, a sample is a rodent sample. In some embodiments, a sample is a non-human primate sample. In some embodiments, a sample is a plant sample. In some embodiments, a sample is a bacterial sample. In some embodiments, a sample is a fungal sample.

### Peptide Tags

Aspects of the present disclosure relate to one or more affinity reagents attached to a peptide tag. In some embodiments, a first affinity reagent is attached to a first peptide tag. In some embodiments, a second affinity reagent is attached to a second peptide tag. In some embodiments, an affinity reagent is attached to a peptide tag. In some embodiments, an affinity reagent is attached to a peptide tag directly or indirectly. In some embodiments, an affinity reagent is attached to a peptide tag via a linker. In some embodiments, a linker is a peptide linker. In some embodiments, a linker is an oligonucleotide linker. In some embodiments, an affinity reagent is attached to a peptide tag via a peptide linker. In some embodiments, an affinity reagent is attached to a peptide tag via an oligonucleotide linker. In some embodiments, a peptide tag is attached to a site on an affinity reagent other than a site on the affinity reagent that binds to a target analyte. In some embodiments, an affinity reagent is an antibody and a peptide tag is attached to the Fc region of the antibody. In some embodiments, a peptide tag is attached covalently to an affinity reagent. In some embodiments, a peptide tag is attached non-covalently to an affinity reagent.

In some embodiments, the term “peptide tag” refers to a peptide comprising one or more amino acids. In some embodiments, a peptide tag is up to 200 amino acids in length (e.g., 5-200, 10-100, 5-80, 5-50, 5-30, 5-20, 20-100, 20-80, or 30-70 amino acids in length). In some embodiments, a peptide tag comprises a cleavage site. In some embodiments, a peptide tag comprises one or more peptide barcodes. In some embodiments, a peptide tag comprises a functional moiety. A peptide tag, as described herein, is the information unit of the affinity reagent. If a target analyte is present within a sample, an affinity reagent attached to a peptide tag can bind to the target analyte, at which point the peptide tag, or a portion thereof, can be sequenced to determine the identity of the target analyte and thus to determine the presence or absence of the target analyte. A peptide tag is specific to the affinity reagent to which it is attached, which is in turn specific to a target analyte. In some embodiments, two affinity reagents are used to bind to a target analyte. If both affinity reagents bind to a target analyte, then the peptide tags attached to each affinity reagent interact with each other through their functional moieties. In some embodiments, a first peptide tag binds to a second peptide tag. If a first peptide tag and a second peptide tag bind to each other, then one or both peptide tags can be analyzed (i.e., sequenced) to determine the identity of the target analyte. In some embodiments, a first peptide tag modifies a second peptide tag. If a first peptide tag modifies a second peptide tag, then the modified peptide tag can be analyzed (i.e., sequenced) to determine the identity of a target analyte.

In some embodiments, a peptide tag, as described herein, comprises a cleavage site. In some embodiments, a cleavage site is a site on a peptide tag that is configured to be cleaved, thus separating the peptide tag from the affinity reagent. Upon separation from an affinity reagent, a peptide tag, or a portion thereof, can be further analyzed (i.e., sequenced) to determine the identity of the target analyte. In some embodiments, the cleavage site is an amino acid or sequence of amino acids in the peptide tag. In some embodiments, the cleavage site of a peptide tag is located between a peptide barcode and the site of attachment between the peptide tag and an affinity reagent. In this way, cleavage at the cleavage site separates the peptide tag from the affinity reagent while retaining the barcode information in the peptide tag (e.g., for analysis by sequencing). In some embodiments, the peptide tag is cleaved by a cleaving agent. In some embodiments, a peptide tag is cleaved prior to further analysis (i.e., sequencing). In some embodiments, the peptide tag is cleaved by a cleaving enzyme. In some embodiments, a cleaving enzyme is a protease, such as an endopeptidase. An endopeptidase is a proteolytic peptidase that breaks peptide bonds of nonterminal amino acids. Non-limiting examples of endopeptidases and corresponding cleavage sites suitable for use in accordance with the disclosure include, without limitation: endoproteinase LysC (cuts after lysine), trypsin (cuts after arginine or lysine, unless followed by a proline), chymotrypsin (cuts after phenylalanine, tryptophan, or tyrosine, unless followed by a proline), elastase (cuts after alanine, glycine, serine, or valine, unless followed by a proline), thermolysin (cuts before isoleucine, methionine, phenylalanine, tryptophan, tyrosine, or valine, unless preceded by a proline), pepsin (cuts before leucine, phenylalanine, tryptophan, or tyrosine, unless preceded by a proline), glutamyl endopeptidase (cuts after glutamate), neprilysin, and prolyl endopeptidase. The structure and function of endopeptidases and endopeptidase cleavage sites are known in the art. For example, endopeptidases are described in van der Velden VH, Hulsmann AR. Peptidases: structure, function and modulation of peptide-mediated effects in the human lung. Clin Exp Allergy. 1999 April; 29(4):445-56, the entirety of which is hereby incorporated by reference.

In some embodiments, a peptide tag, as described herein, comprises one or more peptide barcodes. In some embodiments, the term “peptide barcode” refers to a peptide, or a string of amino acids, that encodes information that is specific to the affinity reagent, the peptide barcode itself, and/or the sample containing a target analyte. In some embodiments, a peptide barcode comprises a reagent barcode (i.e., a barcode that is specific to the affinity reagent). In some embodiments, a peptide barcode comprises a unique molecular identifier (UMI) (i.e., a barcode that is specific to the peptide tag). In some embodiments, a peptide barcode comprises a sample barcode (i.e., a barcode that is specific to the sample containing a target analyte). In some embodiments, a peptide barcode comprises a reagent barcode, a

UMI, and a sample barcode. In some embodiments, a peptide barcode comprises a reagent barcode and a UMI. In some embodiments, a peptide barcode comprises a reagent barcode and a sample barcode. In some embodiments, a peptide barcode comprises a UMI and a sample barcode. In some embodiments, a peptide barcode is at least 1, at least 2, at least 3, at least 4, at least 5, at least 6, at least 7, at least 8, at least 9, at least 10, at least 15, at least 20, at least 25, at least 30, at least 35, at least 40, at least 45, or at least 55 amino acid residues in length. In some embodiments, the peptide barcode is between about 5 and about 50 (e.g., 5-30, 5-20, 10-50, 15-40, 20-40, 20-30, or 15-25) amino acids in length. Once an affinity reagent binds to a target analyte, the peptide tag attached to the affinity reagent can be cleaved from the affinity reagent and the peptide tag, or a portion thereof (e.g., the peptide barcode) can be analyzed (i.e., sequenced) to determine the identity of the target analyte.

In some embodiments, a peptide tag, as described herein, comprises a functional moiety. In some embodiments, the term “functional moiety” refers to a section of a peptide tag that interacts with (e.g., binds to) or is configured to interact with another functional moiety, that is modified or configured to be modified by another functional moiety, or that modifies or is configured to modify another functional moiety. In some embodiments, a first affinity reagent attached to a peptide tag binds to a target analyte and a second affinity reagent attached to a peptide tag binds to the target analyte. Once both affinity reagents are bound to a target analyte, functional moieties on each peptide tag can interact with each other. In some embodiments, a first functional moiety binds to a second functional moiety. In some embodiments, a first functional moiety is configured to be attached to a second functional moiety. In some embodiments, a first functional moiety binds covalently to a second functional moiety. In some embodiments, a first functional moiety binds non- covalently to a second functional moiety.

In some embodiments, a first functional moiety and a second functional moiety each comprises a click chemistry handle. In some embodiments, a first functional moiety and a second functional moiety comprise complementary click chemistry handles.

In some embodiments, the phrase “click chemistry handle” refers to a chemical composition, such as a reactant or a reactive group, that participates in a click chemistry reaction. For example, a strained alkyne, e.g., a cyclooctyne, is a click chemistry handle, since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein, for example, in Tables 1 and 2. In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Additional suitable click chemistry handles are well known to those of skill in the art, and such click chemistry handles include, but are not limited to, the click chemistry reaction partners, groups, and handles described in Becer, Hoogenboom, and Schubert, Click Chemistry beyond Metal-Catalyzed Cycloaddition, Angewandte Chemie International Edition (2009) 48: 4900-4908 and PCT/US2012/044584 and references therein, which references are incorporated herein by reference for click chemistry handles and methodology.

TABLE 1
Exemplary click chemistry handles and reactions.

| | |
|---|---|
| 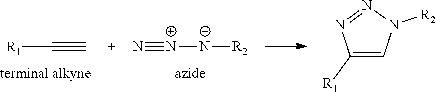 | 1,3-dipolar cycloaddition |
| 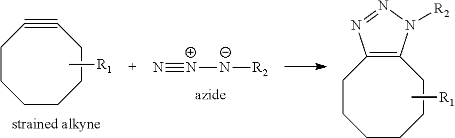 | Strain-promoted cycloaddition |
| 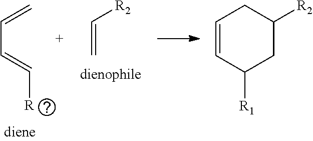 | Diels-Alder reaction |
| 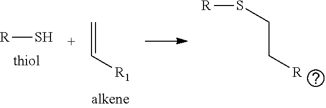 | Thiol-one reaction |

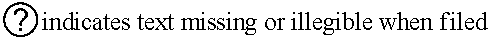

TABLE 2
Exemplary click chemistry handles and reactions (from Becer, Hoogenboom, and
Schubert, Click Chemistry Beyond Metal-Catalyzed Cycloaddition,
Angewandte Chemie International Edition (2009) 48: 4900-4908.).

| Reagent A | Reagent B | Mechanism | Notes on reaction^{[a]} | Reference |
|---|---|---|---|---|
| azide | alkyne | Cu-catalyzed [3 + 2] azide- | 2 h at 60° C. in H₂O | [9] |
| | | alkyne cycloaddition | | |
| | | (CuAAC) | | |
| azide | cyclooctyne | strain-promoted [3 + 2] | 1 h at RT | [6-8, |
| | | azide-alkyne cycloaddition | | 10, 11] |
| | | (SPAAC) | | |
| azide | activated | [3 + 2] Huisgen | 4 h at 50° C. | [12] |
| | alkyne | cycloaddition | | |
| azide | electron- | [3 + 2] cycloaddition | 12 h at RT in H₂O | [13] |
| | deficient | | | |
| | alkyne | | | |
| azide | aryne | [3 + 2] cycloaddition | 4 h at RT in THF with | [14, 15] |
| | | | crown ether or 24 h at | |
| | | | RT in CH₃CN | |
| tetrazine | alkene | Diels-Alder retro-[4 + 2] | 40 min at 25° C. (100% | [36-38] |
| | | cycloaddition | yield) | |
| | | | N₂ is the only by-product | |
| tetrazole | alkene | 1,3-dipolar cycloaddition | few min UV irradiation | [39, 40] |
| | | (photoclick) | and then overnight at | |
| | | | 4° C. | |
| dithioester | diene | hetero-Diels-Alder | 10 min at RT | [43] |
| | | cycloaddition | | |
| anthracene | maleimide | [4 + 2] Diels-Alder | 2 days at reflux in | [41] |
| | | reaction | toluene | |
| thiol | alkene | radical addition | 30 min UV (quantitative | [19-23] |
| | | (thio click) | conv.) or | |
| | | | 24 h UV irradiation | |
| | | | (>96%) | |
| thiol | enone | Michael addition | 24 h at RT in CH₃CN | [27] |
| thiol | maleimide | Michael addition | 1 h at 40° C. in THF or | [24-26] |
| | | | 16 h at RT in dioxane | |
| thiol | para-fluoro | nucleophilic | overnight at RT in DMF | [32] |
| | | substitution | or 60 min at 40° | |
| | | | C. in DMF | |
| amine | para-fluoro | nucleophilic | 20 min MW at 95° C. in | [30] |
| | | substitution | NMP as solvent | |

^{[a]}RT = room temperature, DMF = N,N-dimethylformamide, NMP = N-methylpyrolidone, THF = tetrahydrofuran, CN₃CN = acetonitrile

In some embodiments, the phrase “click chemistry reaction” refers to a group of chemical synthesis reactions that join two molecular entities together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition). Exemplary click chemistry reactions include, but are not limited to, azide-alkyne Huisgen cycloaddition; and Diels-Alder reactions (e.g., tetrazine [4+2] cycloaddition). In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation). In some embodiments, a click chemistry reaction is a Copper(I)-catalyzed reaction. In some embodiments, a click chemistry reaction is a Copper(I)-catalyzed azide-alkyne cycloaddition. In some embodiments, a click chemistry is a copper-free click reaction. In some embodiments, a click chemistry reaction is an azide-alkyne cycloaddition. In some embodiments, a click chemistry reaction is an alkyne-nitrone cycloaddition. In some embodiments, a click chemistry reaction is an alkene/azide cycloaddition. In some embodiments, a click chemistry reaction is an alkene/terazole photoclick reaction. In some embodiments, a click chemistry reaction is any click chemistry reaction known in the art.

In some embodiments, functional moiety binding is indicative of complex formation which is indicative of the presence of a target analyte within a sample. In addition to binding to each other, one functional moiety can modify another functional moiety. In some embodiments, a first functional moiety modifies a second functional moiety. In some embodiments, a first functional moiety is an enzyme and a second functional moiety is an enzyme substrate. In some embodiments, an enzyme is a kinase and an enzyme substrate is a phosphorylation site. Kinases catalyze the phosphorylation of peptides, generally by transferring a phosphoryl group from ATP to the side chain of serine, threonine, or tyrosine. Examples of kinases are known in the art and include, without limitation, PI3K, Akt, mTOR, PKC, MAPKs, AMPK, CDKs, and JAK. In some embodiments, a first functional moiety comprises a kinase (e.g., a kinase selected from PI3K, Akt, mTOR, PKC, MAPKs, AMPK, CDKs, and JAK), and a second functional moiety comprises a phosphorylation site (e.g., an amino acid selected from serine, threonine, and tyrosine). In some embodiments, a first functional moiety comprises a glycosyltransferase, and a second functional moiety comprises a glycosylation site. In some embodiments, a first functional moiety comprises a methyltransferase, and a second functional moiety comprises a methylation site. In some embodiments, a first functional moiety comprises an acetyltransferase, and a second functional moiety comprises an acetylation site. Additional examples of suitable enzyme-substrate pairs suitable for use in accordance with the disclosure are known in the art and would be apparent to skilled practitioners in the field. In some embodiments, modification of a substrate functional moiety by an enzyme functional moiety is indicative of complex formation which is indicative of the presence of a target analyte within a sample.

A first functional moiety can also chemically modify a peptide tag that does not have a functional moiety. In some embodiments, a functional moiety within a peptide tag attached to a first affinity reagent chemically modifies a peptide tag attached to a second affinity reagent. In some embodiments, a functional moiety chemically modifies a peptide tag. In some embodiments, a functional moiety chemically modifies a peptide tag in the presence of a target analyte. In some embodiments, a functional moiety modifies an amino acid residue on a peptide tag. In some embodiments, a functional moiety cleaves one or more amino acid residues from a peptide tag. For example, in some embodiments, the first functional moiety of one peptide tag comprises a cleaving enzyme (e.g., an exopeptidase, an endopeptidase), and the second functional moiety of another peptide tag is an amino acid or amino acid sequence capable of being cleaved by the cleaving enzyme. In some embodiments, the first functional moiety of one peptide tag comprises an exopeptidase (e.g., an aminopeptidase, a carboxypeptidase), and the second functional moiety of another peptide tag is an amino acid or amino acid sequence capable of being cleaved by the exopeptidase. The structure and function of exopeptidases and exopeptidase cleavage sites are known in the art. For example, exopeptidases (e.g., aminopeptidases, carboxypeptidases) and their corresponding cleavage sites are described in: PCT International Publication Nos. WO2020/102741A1, WO2021/236983A2, WO2023/122769A2, WO2024/031031A2, WO2024/086832A1, and WO2025/147658A1; and van der Velden VH, Hulsmann AR. Peptidases: structure, function and modulation of peptide-mediated effects in the human lung. Clin Exp Allergy. 1999 April; 29(4):445-56, the entirety of each of which is hereby incorporated by reference. In some embodiments, chemical modification of a peptide tag is indicative of complex formation which is indicative of the presence of a target analyte within a sample.

Complex formation (i.e., a first affinity reagent and a second affinity reagent each binding to a target analyte or a single affinity reagent binding a target analyte) is indicative of the presence of a target analyte within a sample. Once a complex forms, peptide tags can be cleaved from the affinity reagents and analyzed (i.e., sequenced) by peptide barcode sequencing.

### Peptide Barcode Sequencing

Aspects of the present disclosure relate to methods comprising protein sequencing, e.g., sequencing of peptide barcodes. A peptide barcode, as described herein, is an amino acid sequence within a peptide tag. In some embodiments, a nucleic acid encoding an affinity reagent appended to a peptide tag containing a peptide barcode is expressed in a cell or within a sample. In some embodiments a nucleic acid encoding an affinity reagent appended to a peptide tag containing a peptide barcode is expressed in vitro or in vivo. In some embodiments, the nucleic acid is expressed as an expression cassette. In some embodiments, a peptide tag is cleaved from an affinity reagent and only the peptide tag, comprising a peptide barcode, is prepared for sequencing and sequenced. In some embodiments, a peptide barcode is cleaved from a peptide tag and only the peptide barcode is prepared for sequencing and sequenced. In some embodiments, the peptide barcode is cleaved by a cleaving agent. In some embodiments, the peptide barcode is at least 1, at least 2, at least 3, at least 4, at least 5, at least 6, at least 7, at least 8, at least 9, at least 10, at least 15, at least 20, at least 25, at least 30, at least 35, at least 40, at least 45, or at least 55 amino acid residues in length. In some embodiments, the peptide barcode is between about 5 and about 50 (e.g., 5-30, 5-20, 10-50, 15-40, 20-40, 20-30, or 15-25) amino acids in length. In some embodiments, the peptide barcode produces a signature trace when sequenced, that allows a user to correlate the peptide barcode with the peptide tag and affinity reagent to which it was attached. In some embodiments, the presence or absence of a target analyte is determined by sequencing the peptide barcode. In some embodiments, the identity of a target analyte is determined by sequencing the peptide barcode. In some embodiments, the abundance of a protein is quantified by sequencing the peptide barcode.

Aspects of the present disclosure relate to preparing a peptide barcode library using an enzymatic reaction. In some embodiments, a protein construct is produced comprising tags and cleavage sites for immobilizing, cleaving, and isolating a peptide barcode. In some embodiments, a tag is used to immobilize a protein construct onto a microbead. In some embodiments, the microbead is a “nickel-charged affinity resin.” In some embodiments, the enzyme is a Sortase. In some embodiments, the enzyme is Sortase A. In some embodiments, a His-tag is used on the protein construct to isolate or purify the peptide barcode after library preparation.

In some embodiments, a peptide barcode comprises the sequence “LPETGG” (SEQ ID NO: 1) which is recognized by an enzyme (e.g., sortase) described herein. In some embodiments, the enzyme adds an enzyme nucleophile to the peptide barcode. In some embodiments, the enzyme nucleophile is triglycine-PEG3-Picolyl azide, triglycine-Lys(N3) (SEQ ID NO: 2), or 3-azido-1-propylamine. In some embodiments, a triglycine-azide is added to the peptide barcode at the “LPETGG” (SEQ ID NO: 1) sequence. In some embodiments, the azide is available for a further chemical reaction. In some embodiments, the further chemical reaction is a click chemistry reaction. In some embodiments, the click chemistry reaction is a copper-free click reaction. In some embodiments, after library preparation, the peptide barcode is purified or isolated for peptide sequencing.

Aspects of the present disclosure relate to a method to design amino acid sequences (“peptide barcodes”) to be used in single-molecule protein sequencing applications. In some embodiments, peptide barcodes contain one or more “information” regions and “functional” regions. In some embodiments, peptide barcodes contain only an “information” region. In some embodiments” the information region is used for peptide sequencing. In some embodiments, the functional region is used for further library preparation. In some embodiments, peptide barcodes give rise to Regions of Interest (ROIs) when sequenced. In some embodiments, an ROI represents one or more amino acid residues. ROIs stem from the on-off binding of a recognizer to the N-terminal amino acid. Transitions between ROIs depend on enzymatic cutting of the N-terminal amino-acid resulting in the exposure of a different N-terminal amino acid. Recognizers have multiple substrates, a single recognizer can bind several N-terminal amino-acids. In some embodiments, the recognizer is PS1223, PS1220, PS610, PS1259, or PS1165. In some embodiments, PS1223 recognizes leucine (L), isoleucine (I), and/or valine (V). In some embodiments PS1220 recognizes arginine (R). In some embodiments, PS610 recognizes phenylalanine (F), tyrosine (Y), and/or tryptophan (W). In some embodiments, PS1259 recognizes glutamine (Q) and/or asparagine (N). In some embodiments, PS1165 recognizes alanine (A) and/or serine (S). Additional amino acid recognizers suitable for peptide sequencing have been described and characterized, for example, in PCT International Publication Nos. WO2020/102741A1, WO2021/236983A2, WO2023/122769A2, WO2024/031031A2, WO2024/086832A1, and WO2025/147658A1, each of which is incorporated by reference in its entirety.

A peptide barcode signal is interpreted via its ROIs. Each ROI is generated by one of the recognizers used in sequencing the peptide barcode (e.g., one of at least two, three, four, five, or more recognizers), labeled with a specific dye, that emits light with specific properties. In some embodiments, the dye is luminescent. In some embodiments, the dye is a fluorophore. Within an ROI consistent pulsing by one recognizer is observed. In some embodiments, each pulse in the ROI has a Pulse Width (PW) and an Inter Pulse Distance (IPD). The median pulse width and the median IPD are distinctive of the ROIs and sensitive to the nature of the N-terminal amino-acid and of the context (penultimate amino-acid and beyond). The signal from one aperture is often idealized as a ‘kinetic signature’ that summarizes the properties of the ROIs. Two properties that are typically used are ‘color’ distinguishing the different recognizers and median PW (as in the example here). Other properties can include, e.g., median IPD.

In some embodiments, the pulsing properties of each residue along the amino acid barcode (ROIs) can be analyzed on a substrate (e.g., an array, a chip). ROIs are described by multiple parameters including binratio (average or distribution), pulse width (average or distribution), interpulse distance (average or distribution). In some embodiments, the ROI is described by binratio, pulse width, an interpulse distance. In some embodiments, the ROI is described by binratio only. In some embodiments, the ROI is described by pulse width only. In some embodiments, the ROI is described by interpulse distance only. In some embodiments, ROI are discretized based on mean binratio and mean pulse width. In some embodiments, peptide barcodes with 5 ROIs give a total diversity of 625 possible barcodes (5*5*5*5). In some embodiments, barcode identity is mapped to its ROIs. In some embodiments, the abundance of a protein of interest can be quantified by sequencing peptide barcodes are analyzing peptide barcode ROIs.

### Polypeptide Analysis

In some aspects, the disclosure provides methods of polypeptide analysis (e.g., polypeptide sequencing) to sequence a peptide barcode. In some embodiments, a method of polypeptide analysis comprises: contacting a polypeptide with one or more amino acid recognizers (e.g., a reaction mixture comprising one or more amino acid recognizers) described herein; monitoring a signal for signal pulses corresponding to interactions between one or more amino acid recognizers (e.g., amino acid binding proteins) and the polypeptide; and determining at least one chemical characteristic of the polypeptide based on a characteristic pattern in the signal.

Compositions and methods for analyzing (e.g., sequencing) peptides in accordance with the disclosure have been described, for example, in PCT International Publication Nos. WO2020/102741A1, WO2021/236983A2, WO2023/122769A2, WO2024/031031A2, WO2024/086832A1, and WO2025/147658A1, each of which is incorporated by reference in its entirety.

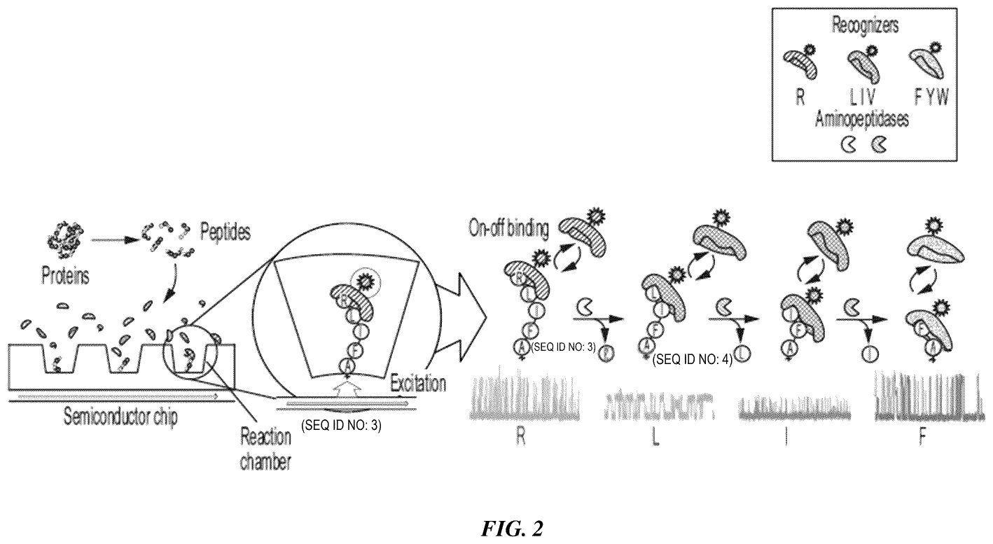

A non-limiting example of polypeptide structure analysis by detecting single molecule binding interactions during a polypeptide degradation process is illustrated in FIG. 2. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. In some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 2, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 2, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the disclosure are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the disclosure are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the disclosure. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

In some embodiments, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. In some embodiments, an array comprises millions of sample wells. In some embodiments, an array comprises at least 1,000,000, at least 2,000,000, at least 3,000,000, at least 4,000,000, at least 5,000,000, at least 6,000,000, at least 7,000,000, at least 8,000,000, at least 9,000,000, at least 10,000,000, at least 15,000,000, at least 20,000,000, at least 25,000,000, or at least 30,000,000 sample wells. In some embodiments, an array comprises at least 8,000,000 to at least 32,000,000 million sample wells. In some embodiments, an array comprises 2,000,000 sample wells. In some embodiments, an array comprises 8,000,000 sample wells. In some embodiments, an array comprises 32,000,000 sample wells. The volume of a sample well may be between about 10⁻²¹ liters and about 10⁻¹⁵ liters , in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

### Methods

Aspects of the present disclosure relate to a method of identifying a target analyte within a sample. Specifically, aspects of the present disclosure relate to use of any of the compositions or complexes described herein to identify a target analyte within a sample. In some embodiments, a method of identifying a target analyte within a sample comprises: (i) contacting a sample with a first affinity reagent attached to a first peptide tag and a second affinity reagent attached a to a second peptide tag; (ii) contacting the sample with a cleaving agent; and (iii) sequencing at least a portion of the first peptide tag and/or at least a portion of the second peptide tag. A peptide tag is any peptide tag described herein. In some embodiments, the cleaving agent removes the first peptide tag from the first affinity reagent and/or the second peptide tag from the second affinity reagent.

Sequencing of the first peptide tag and/or the second peptide tag can occur within the sample (i.e., in situ) or outside the sample. In some embodiments, the method further comprises isolating the first peptide tag and/or the second peptide tag from the sample prior to sequencing.

As described herein, a first functional moiety within a first peptide tag can interact with a second functional moiety within a second peptide tag. In some embodiments, a first functional moiety binds to a second functional moiety. In some embodiments, a first peptide tag is ligated to a second peptide tag. Accordingly, in some embodiments, the method further comprises contacting a sample with a peptide ligase. In some embodiments, the phrase “peptide ligase” refers to an enzyme that can link (e.g., cross-link) two peptides together. In some embodiments, a peptide ligase links a first peptide tag to a second peptide tag. In some embodiments, a peptide ligase is a cysteine protease with ligase activity. In some embodiments, a cysteine ligase is a sortase. In some embodiments, a cysteine ligase is a butelase I. In some embodiments, a peptide ligase is an engineered peptide ligase. In some embodiments, an engineered peptide ligase is trypsiligase. In some embodiments, an engineered peptide ligase is subtiligase. Examples of ligases and corresponding ligatable sequences suitable for use in accordance with the disclosure would be apparent to skilled practitioners in the field based on knowledge in the art. Table 3 provides non-limiting examples of such ligases and ligation reactions.

TABLE 3
Exemplary ligases and reactions (adapted from Goettig, P. Reversed
Proteolysis-Proteases as Peptide Ligases. Catalysts 2021, 11,
33, the entirety of which is hereby incorporated by reference.).

| | Ligated | Example reaction | |
|---|---|---|---|
| Protease/Ligase | bond | conditions | Source |
| α-Chymotrypsin | Pro- | H₂O, pH 8.0, | Günther, R., et al. Eur. |
| | Arg | 10% MeOH | J. Biochem. 2000, 267, |
| | | | 3496-3501. |
| α-Chymotrypsin | Tyr- | H₂O (−15° C.) | Wehofsky, N., et al. |
| | Gly | | Tetrahedron Asymmetry |
| | | | 2000, 11, 2421-2428. |
| β-Trypsin | Arg- | DMF/HFIP 1 1:1, | Nishino, N., et al. J. |
| | Lys | 4% H₂O | Chem. Soc. Chem. |
| | | | Commun. 1992, 10, |
| | | | 648-650. |
| β-Trypsin | Arg- | H₂O (−15° C.) | Wehofsky, N., et al. |
| | Ala | | Tetrahedron Asymmetry |
| | | | 2000, 11, 2421-2428. |
| Trypsiligase | Arg- | H₂O, pH 7.8 | Liebscher, S., et al. |
| (rat) | His | | ChemBioChem 2014, |
| | | | 15, 1096-1100. |
| Elastase-1 | Leu- | H₂O, pH 9.0 | Haensler, M., et al. Biol. |
| (porcine) | Ala | (NaOH) | Chem. 1998, 379, 71- |
| | | | 74. |
| Papain | Phe- | H₂O, 0.2M K₂HPO₄, | Haensler, M., et al. J. |
| | Leu | pH 7.8 | Pept. Sci. 1996, 2, 279- |
| | | | 289. |
| Pepsin | Phe- | H₂O, pH 4.5, | Morihara, K., et al. J. |
| | Leu | 10% DMF | Biochem. 1981, 89, |
| | | | 385-395. |
| Carboxypeptidase B | Tyr- | H₂O, pH 6.7 | Sealock, R. W., et al. |
| | Lys | | Biochemistry 1969, 8, |
| | | | 3703-3710. |
| Thermolysin | Phe- | H₂O, pH 7.5, | Wayne, S. I., et al. Proc. |
| | Phe | 20% DMSO | Natl. Acad. Sci. USA |
| | | | 1983, 80, 3241-3244. |
| Subtilisin | Phe- | DMF/H₂O 1:1 | Barbas, C. F., et al. J. |
| BPN′ | Leu | | Am. Chem. Soc. 1988, |
| | | | 110, 5162-5166. |
| Thiolsubtilisin C | Phe- | H₂O, pH 8.0, | Nakatsuka, T., et al. J. |
| | Val | 40% DMF | Am. Chem. Soc. 1987, |
| | | | 109, 3808-3810. |
| Selenosubtilisin C | Cin- | H₂O, 0.1M borate, | Wu, Z. P., et al. J. Am. |
| | Gly | pH 9.3 | Chem. Soc. 1989, 111, |
| | | | 4513-4514. |
| Subtiligase | Phe- | H₂O, pH 8.0 | Abrahmsen, L., et al. |
| BPN′ | Ala | | Biochemistry 1991, 30, |
| | | | 4151-4159. |
| Thymoligase | Lys- | H₂O, phosphate | Schmidt, M., et al. Org. |
| | Asp | pH 8.3 | Biomol. Chem. 2018, |
| | | | 16, 609-618. |
| Sortase A | Thr- | H₂O, phosphate | Nguyen, G. K. T., et al. J. |
| | Gly | pH 6.0 | Am. Chem. Soc. 2015, |
| | | | 137, 15398-15401. |
| γ-Legumain | Asn- | H₂O, phosphate | Nguyen, G. K. T., et al. J. |
| (plants) | Gly | pH 6.0 | Am. Chem. Soc. 2015, |
| | | | 137, 15398-15401. |

The present disclosure is related, at least in part, to a method of identifying a target analyte in a sample, comprising contacting a sample with any composition or complex described herein. The present disclosure is also related to a system to perform any method described herein. In some embodiments, a system, as described herein, comprises at least one hardware processor and at least one non-transitory computer-readable storage medium storing processor-executable instructions that, when executed by the at least one hardware processor, cause the at least one hardware processor to perform any method described herein. The present disclosure is also related to at least one non-transitory computer-readable storage medium storing processor-executable instructions that, when executed by at least one hardware processor, cause the at least one hardware processor to perform any method described herein.

### Devices and Systems

Methods in accordance with the disclosure, in some aspects, may be performed using a system that permits sequencing of a peptide barcode described herein. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may case delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to other optical components of the integrated device and direct the excitation light to the other optical components. For example, the optical system may include optical components that direct the excitation light from the grating coupler(s) towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an integrated device may be configured to perform single-molecule analysis in combination with an instrument as described above. It should be appreciated that the integrated device described herein is intended to be illustrative and that other integrated device configurations may be configured to perform any or all techniques described herein.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the sample well. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### Kits

Aspects of the present disclosure relate to a kit, comprising materials and reagents for carrying out any method described herein. A kit, as described herein, comprises, for example, a first affinity reagent attached to any peptide tag described herein and/or a second affinity reagent attached to any peptide tag described herein. In some embodiments, a kit comprises an affinity reagent attached to a peptide tag comprising a cleavage site, one or more peptide barcodes, and a functional moiety. In some embodiments, a kit comprises an affinity reagent attached to a peptide tag comprising a cleavage site and one or more peptide barcodes. In some embodiments, a kit comprises one or more affinity reagents, each attached to a peptide tag, materials and reagents for carrying out any method described herein, and instructions for use.

### Equivalents And Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A composition comprising:
a first affinity reagent attached to a first peptide tag; and
a second affinity reagent attached to a second peptide tag;
wherein the first affinity reagent and the second affinity reagent are each configured to bind a target analyte to form a complex comprising the first affinity reagent, the second affinity reagent, and the target analyte.

2. The composition of claim 1, wherein the first affinity reagent is directly attached to the first peptide tag.

3. The composition of claim 1, wherein the second affinity reagent is directly attached to the second peptide tag.

4. The composition of claim 1, wherein the first affinity reagent is attached to the first peptide tag via a linker, optionally wherein the linker is a peptide linker or an oligonucleotide linker.

5. The composition of claim 1, wherein the second affinity reagent is attached to the second peptide tag via a linker, optionally wherein the linker is a peptide linker or an oligonucleotide linker.

6. (canceled)

7. The composition of claim 1, wherein the target analyte is a target protein, optionally wherein the target protein is a monomeric or multimeric protein.

8. (canceled)

9. The composition of claim 1, wherein the first affinity reagent and the second affinity reagent are each configured to bind different sites on the target analyte.

10. (canceled)

11. The composition of claim 1, wherein the first affinity reagent is an antibody, a nanobody, an aptamer, or an adaptor protein and/or wherein the second affinity reagent is an antibody, a nanobody, an aptamer, or an adaptor protein.

12. (canceled)

13. The composition of claim 1, wherein the first peptide tag comprises a first cleavage site, one or more first peptide barcodes, and/or a first functional moiety and/or wherein the second peptide tag comprises a second cleavage site, one or more second peptide barcodes, and/or a second functional moiety.

14. (canceled)

15. The composition of claim 13, wherein the one or more first peptide barcodes comprise a reagent barcode, a unique molecular identifier, a sample barcode, or any combination thereof, and/or wherein the one or more second peptide barcodes comprise a reagent barcode, a unique molecular identifier, a sample barcode, or any combination thereof.

16. (canceled)

17. The composition of claim 13, wherein the first functional moiety is a click chemistry handle, an enzyme, or an enzyme substrate, and/or wherein the second functional moiety is a click chemistry handle, an enzyme, or an enzyme substrate.

18.-19. (canceled)

20. The composition of claim 1, wherein the first peptide tag comprises a first functional moiety, optionally wherein the first functional moiety chemically modifies the second peptide tag, further optionally wherein the first functional moiety chemically modifies the second peptide tag in the presence of the target analyte.

21.-22. (canceled)

23. The composition of claim 20, wherein the first functional moiety chemically modifies the second peptide tag when the first affinity reagent and the second affinity reagent are each bound to the target analyte.

24. The composition of claim 20, wherein the first functional moiety chemically modifies an amino acid residue on the second peptide tag.

25. The composition of claim 20, wherein the first functional moiety cleaves one or more amino acid residues from the second peptide tag.

26. The composition of claim 20, wherein the first functional moiety comprises an enzyme, and the second peptide tag comprises a substrate of the enzyme.

27. The composition of claim 20, wherein the second peptide tag comprises a second functional moiety.

28. The composition of claim 27, wherein the first functional moiety comprises an enzyme, and the second functional moiety comprises a substrate of the enzyme or wherein the first functional moiety and the second functional moiety comprise complementary click chemistry handles.

29.-35. (canceled)

36. A complex, comprising:
a first affinity reagent attached to a first peptide tag; and
a second affinity reagent attached to a second peptide tag;
wherein the first affinity reagent and the second affinity reagent are each bound to a target analyte.

37.-66. (canceled)

67. A method of identifying a target analyte, comprising:
(i) contacting a sample with one or more analyte-specific reagent sets, each analyte-specific reagent set comprising a first affinity reagent attached to a first peptide tag and a second affinity reagent attached to a second peptide tag, wherein the first affinity reagent and the second affinity reagent are each configured to bind a target analyte;
(ii) contacting the sample with a cleaving agent, wherein the cleaving agent removes the first peptide tag from the first affinity reagent and/or the second peptide tag from the second affinity reagent; and
(iii) sequencing at least a portion of the first peptide tag and/or at least a portion of the second peptide tag.

68.-115. (canceled)


SEQUENCE LISTING (4 sequences)

SEQ ID NO: 1 - protein, 6 aa, synthetic construct
LPETGG

SEQ ID NO: 2 - protein, 4 aa, synthetic construct
  SITE 4: note = Azido-Lysine
GGGK

SEQ ID NO: 3 - protein, 5 aa, synthetic construct
RLIFA

SEQ ID NO: 4 - protein, 4 aa, synthetic construct
LIFA